(19) United States
(12) Patent Application Publication
(10) Pub. No.: US 2020/0337610 A1
(43) Pub. Date: Oct. 29, 2020

(54) APPARATUS AND METHOD FOR NON-INVASIVELY MONITORING BLOOD GLUCOSE

(71) Applicants: Wenwei YU, Chiba (JP); Singapore University of Technology and Design, Singapore (SG)

(72) Inventors: Wenwei YU, Chiba (JP); Shaoying HUANG, Singapore (SG); - OMKAR, Singapore (SG)

(21) Appl. No.: 16/764,828

(22) PCT Filed: Nov. 14, 2018
(86) PCT No.: PCT/SG2018/050570

§ 371 Date: May 15, 2020

(30) Foreign Application Priority Data
Nov. 15, 2017 (SG) 10201709405T

(51) Int. Cl. A61B 5/145; A61B 5/00; A61B 5/053

(52) U.S. Cl. CPC A61B 5/14532; A61B 5/6824; A61B 5/6826; A61B 2560/0228; A61B 5/0537; A61B 2562/0295; A61B 5/6829

(57) ABSTRACT

A non-invasive glucose monitoring apparatus comprises at least one microstrip transmission line (MLIN) component comprising: a microstrip conductor that is arranged relative to a ground plane such that a body part of a user, such as a finger or wrist, is receivable in a space defined between the microstrip conductor and the ground plane, the microstrip transmission line component having an input port; a signal input component for transmitting an input signal to the input port; and a concentration determining component configured to: determine at least one parameter of an output signal of the microstrip transmission line component; and determine, based on a comparison of the at least one parameter to at least one respective calibration curve, a glucose concentration of the user.

### TECHNICAL FIELD

The present invention relates to an apparatus and method for non-invasively monitoring blood glucose.

### BACKGROUND

The prevalence of diabetes has increased rapidly in recent years such that it has become a leading cause of death worldwide. Although there is no cure for diabetes, blood glucose monitoring combined with appropriate medication can enhance treatment efficiency, alleviate the symptoms, and diminish complications.

Typically, glucose meters are electrochemical and require blood samples as input. Electrochemical glucose meters are accepted as being the most accurate and reliable blood glucose measurement devices, but because they rely on a finger-prick mechanism, they are invasive, painful to the user, and eventually result in damage to the nerve system of the patient after long term usage. In addition, diabetic patients may need to conduct six measurements daily, one before and one after each meal.

Due to the disadvantages of invasive blood glucose measurements, some non-invasive monitoring approaches have been investigated. These are primarily aimed at patient comfort, but may also offer the possibility of continuous blood glucose level monitoring, which provides real time information on the condition of the patient (e.g. hypoglycemic and hyperglycemic states) enabling timely guidance on diet and appropriate medical treatments.

A number of approaches for non-invasive glucose monitoring have previously been proposed, including optical, electrochemical, transdermal and microwave/RF techniques.

For example, in the optical category, a wide range of technologies has been applied, including using mid-infrared light, Raman spectroscopy, fiber optics, surface plasmon resonance interferometry, and absorption spectroscopy. These are suitable only for intermittent monitoring as they are typically bulky and unwieldy to set up, and thus not wearable so as to be used for continuous monitoring.

In some other non-invasive approaches, the target for sensing may introduce difficulties if continuous monitoring is desired. For example, one known device measures glucose level by analyzing metabolites in the breath of a subject who blows into a breathalyzer. This presents obvious difficulties for continuous monitoring.

Another type of known device uses the fringing field of a microstrip transmission line (MLIN) to form a capacitor with the object under sensing, namely the skin of the subject. This type of device is called a capacitive fringing-field sensor. It relies on the measurement of the changes of impedance on the dermis layer of the skin through the interference that is captured by the fringing fields of the MLIN. MLIN-based impedance spectroscopy that makes use of the fringing field relies on the fact that the change of the glucose level in blood alters the electrical properties (permittivity and conductivity) of the tissues at the target site. It has been found previously that the sensitivity of MLIN-based sensors is typically low, due to low penetration depth of the fringing fields. Additionally, variation in factors other than glucose level, such as body temperature and hydration, can contribute to the change of electrical properties at the target site.

One way to address the aforementioned problems is to use a MLIN-based sensor in conjunction with other sensors, such as sweat sensors, temperature sensors and the like, in a multi-sensing system for glucose monitoring. Although crosschecking in this fashion may help to increase the sensing accuracy, increasing the number of sensors increases the physical size of the monitoring system and introduces additional sources of errors and interference to the system.

It would be desirable to provide a glucose monitoring device and method that addresses or alleviates one or more of the above difficulties, or which at least provides a useful alternative.

### SUMMARY

In a first aspect, the present disclosure relates to a non-invasive glucose monitoring apparatus, comprising:
at least one microstrip transmission line component comprising a microstrip conductor that is arranged relative to a ground plane such that a body part of a user is receivable in a space defined between the microstrip conductor and the ground plane, the microstrip transmission line component having an input port;
a signal input component for transmitting an input signal to the input port; and
a concentration determining component configured to:
   determine at least one parameter of an output signal of the microstrip transmission line component; and
   determine, based on a comparison of the at least one parameter to at least one respective calibration curve, a glucose concentration of the user.

The output signal may be a reflected signal measured at the input port, for example.

The microstrip conductor may be patterned, and may for example comprise a plurality of repeating units spaced at regular intervals. Individual units of the pattern may be one or more of: a rectangular element; an interdigitated capacitor; a meander inductor; or a spiral inductor.

In some embodiments, the ground plane may also be patterned, or may be patterned instead of the microstrip conductor.

The at least one wearable transmission line component may be in the form of a ring, a finger stall, a bracelet and/or an anklet.

In some embodiments, an output port of the microstrip transmission line component is terminated via a load. The load may be an open circuit, a short circuit, an impedance-matched load, a capacitive load or an inductive load.

The at least one parameter may comprise at least one parameter derived from the input impedance and/or the reflection coefficient. For example, the at least one parameter may comprise one or more of: the real part of the input impedance, the imaginary part of the input impedance, the magnitude of the input impedance, the phase of the input impedance, the real part of the reflection coefficient, the imaginary part of the reflection coefficient, the magnitude of the reflection coefficient, and the phase of the reflection coefficient.

In some embodiments, the concentration determining component is configured to determine the glucose concentration based on a plurality of parameters derived from the reflected signal.

In some embodiments, the microstrip transmission line component is supported within a housing. The signal input component may be within, extend from, or be attached to the housing.

The concentration determining component may be in the form of computer-readable instructions stored on non-volatile storage in communication with at least one processor. The non-volatile storage and the at least one processor may be housed within the housing, for example.

In another aspect, the present disclosure provides a method for non-invasively monitoring blood glucose concentration in a subject, comprising:
transmitting, to an input of a microstrip conductor, an input signal, the microstrip conductor being arranged relative to a ground plane to define a space to receive a body part of the subject, the microstrip conductor and the ground plane together functioning as a microstrip transmission line having the body part of the subject as its substrate;
measuring an output signal from the microstrip transmission line;
determining at least one parameter of the output signal; and
determining, based on a comparison of the at least one parameter to at least one respective calibration curve, a glucose concentration of the user.

The step of measuring the output signal may comprise measuring a reflected signal at the input port, for example.

The at least one parameter may comprise at least one parameter derived from the input impedance and/or the reflection coefficient, for example, one or more of: the real part of the input impedance, the imaginary part of the input impedance, the magnitude of the input impedance, the phase of the input impedance, the real part of the reflection coefficient, the imaginary part of the reflection coefficient, the magnitude of the reflection coefficient, and the phase of the reflection coefficient.

In some embodiments, the glucose concentration may be determined based on a plurality of parameters derived from the output signal.

### BRIEF DESCRIPTION OF THE DRAWINGS

Certain embodiments of the invention will now be described, by way of non-limiting example only, with reference to the accompanying drawings, in which:

FIG. 1 is a schematic depiction of a glucose monitoring apparatus according to certain embodiments;

FIG. 2 shows one configuration of a microstrip transmission line (MLIN) component of a glucose monitoring apparatus;

FIG. 3 shows another configuration of a MLIN component of a glucose monitoring apparatus;

FIG. 4 shows a further configuration of a MLIN component of a glucose monitoring apparatus;

FIG. 5 is a schematic depiction of a MLIN component that has a modulated signal line and a defected ground plane;

FIG. 6 shows three possible repeat units of a modulated signal line according to certain embodiments;

FIG. 7 is a further example of a repeat unit of a modulated signal line according to certain embodiments;

FIG. 8A is a schematic depiction of an experimental setup for testing a MLIN component according to certain embodiments;

FIG. 8B is a schematic depiction of an experimental setup for testing a MLIN component according to the prior art;

FIG. 9 shows measured |S₁₁| as a function of frequency from the test setups of FIGS. 8A and 8B;

FIG. 10 shows measured |S₁₁| at the resonant frequency (left vertical axis) and the resonant frequency (right vertical axis) versus concentration for the test setups of FIGS. 8A and 8B;

FIG. 11 shows measured phase (S₁₁), Re(S₁₁) and Im(S₁₁) for a MLIN component according to certain embodiments;

FIG. 12 shows measured |z₁₁|, phase (z₁₁), Re(z₁₁), Im(z₁₁) for a MLIN component according to certain embodiments;

FIG. 13 shows measured |S₁₁| versus frequency and |S₁₁| sensitivity (a) at 100 MHz-500 MHz with a load of 50Ω, (c) 1 GHz-2 GHz with an open circuit at the load, (e) 1 GHz-2 GHz with a short circuit at the load;

FIG. 14 shows a calibration curve for an exemplary glucose concentration estimation process based on |S₁₁| when the load is 50Ω in the frequency band of 1.4-1.9 GHz;

FIG. 15 shows the estimated error of an estimation process based on (a) single-variate of single parameter (|S₁₁| of an exemplary MLIN), in a single frequency range; (b) single-variate of single parameter (|S₁₁| of an exemplary MLIN), in a single frequency range; (c) multi-variate of a single parameter (the real part, imaginary part, magnitude, and phase of the S₁₁ of an exemplary MLIN), in a single frequency range; and (d) multivariate of multiple parameters (S₁₁ and z₁₁ of an exemplary sub MLIN), in a single frequency range. The load is 50Ω and the frequency range is 1.4-1.9 GHz;

FIG. 16 shows estimated error based on |S₁₁| when the load is 50Ω in two frequency ranges, 1.4-1.9 GHz and 100-500 MHz;

FIG. 17 shows estimated error based on Im(S₁₁) when the load is 50Ω in the frequency range of 1.4-1.9 GHz;

FIG. 18 schematically depicts a test setup for a glucose monitoring apparatus with a patterned microstrip conductor according to certain embodiments;

FIG. 19 shows |S₁₁| versus frequency for the apparatus of FIG. 18 and an unpatterned MLIN counterpart at low and high glucose concentrations;

FIG. 20 shows |S₁₁|ₘᵢₙ versus glucose concentration for the apparatus of FIG. 18 and an unpatterned MLIN counterpart at low and high glucose concentrations;

FIG. 21 shows an exemplary architecture of a processing device of a glucose monitoring apparatus according to certain embodiments; and

FIG. 22 is a flowchart of a method according to certain embodiments.

### DETAILED DESCRIPTION OF EMBODIMENTS

In general terms, embodiments of the present invention relate to a microstrip transmission line (MLIN)-based glucose sensor which is positionable on a subject such that the skin of the subject forms the substrate of the MLIN, i.e. the skin is directly exposed to the main field of the MLIN between the microstrip conductor and the ground plane. Typically, the sensor is wearable, and may be in the form of a ring, finger stall or bracelet, for example. Glucose levels of the subject can be inferred from parameters of an output signal (e.g., the reflected signal) of the transmission line. In this way, the sensor can measure glucose levels non-invasively and on a continuous basis while the sensor is worn. In addition, since the object under sensing is the substrate of the transmission line, it lies in a region where the electromagnetic fields are highly confined, such that the sensitivity of the sensor is increased.

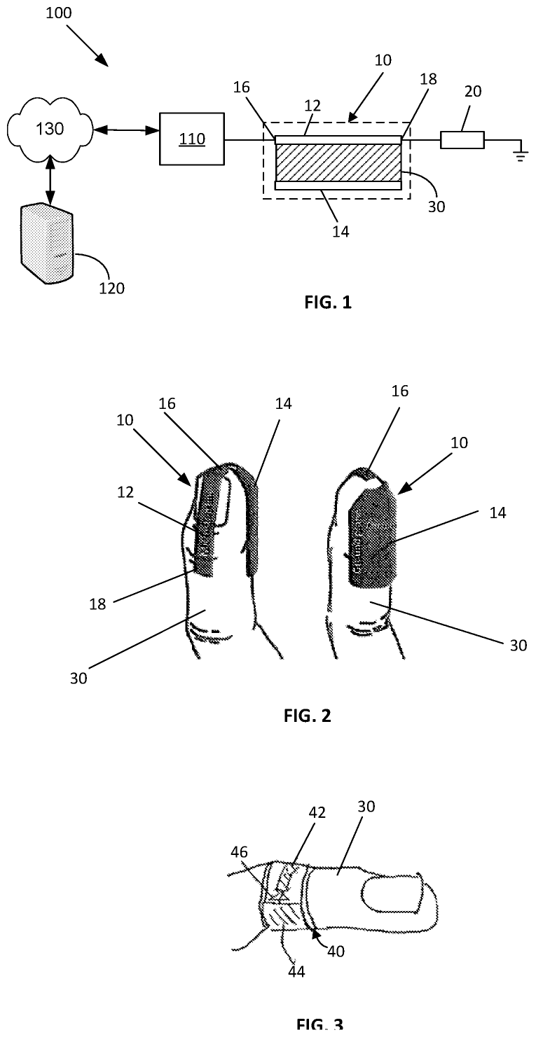

Turning to FIG. 1, there is shown, in schematic form, an example of a glucose monitoring apparatus 100. The glucose monitoring apparatus 100 includes a microstrip transmission line (also referred to herein as a microstrip line or MLIN) component 10 that includes a microstrip conductor 12 spaced from a ground plane 14 such that a body part 30 of a subject can be inserted into the microstrip transmission line component 10, whereby the body part 30 forms a substrate of the microstrip transmission line component 10.

The microstrip conductor may have an input port 16 and an output port 18. The output port 18 may be terminated by a load 20. Each of the input port 16 and output port 18 may comprise an SMA connector for ease of connecting the microstrip conductor to an external device, for example. In some embodiments, the input port 16 and/or the output port 18 may be directly connected to an external circuit without the use of any special connector.

The input port 16 may be coupled to a signal input component 110 for generating and passing an input signal to the MLIN component 10. In some embodiments, the signal input component 110 may also include a signal measurement component for capturing a reflected signal from the transmission line component 10. For example, the signal input component 110 may be a vector network analyser or similar signal generation/measurement device.

The signal input component 110 may be communicatively coupled (for example, via a network 130) to at least one external processor device 120, for example a server computing device that is configured to receive measured reflected signals from the MLIN component 10, to derive one or more parameters from the reflected signals, and to compare the one or more parameters to respective calibration curves in order to estimate a glucose level of the subject, as will later be described in more detail. Thus, the processor device 120 acts as a concentration determining component that is configured to receive output signals from the MLIN component 10, to compare one or more parameters and/or parameter components to one or more calibration curves that are stored in memory of the processor device 120, and to estimate a glucose concentration from the comparison.

While the signal input component 110 and the processor 120 are shown as physically separate components, it will be appreciated that they may be contained within a single housing. For example, the signal generation and measurement functions may be implemented on one printed circuit board (PCB) contained in the housing, with the processor being carried on another PCB. Alternatively, all of the functions of signal input component 110 and processor 120 may be embodied in a single PCB. The housing may have leads extending therefrom to electrically couple the signal input component 110 and/or processor 120 to the MLIN 10.

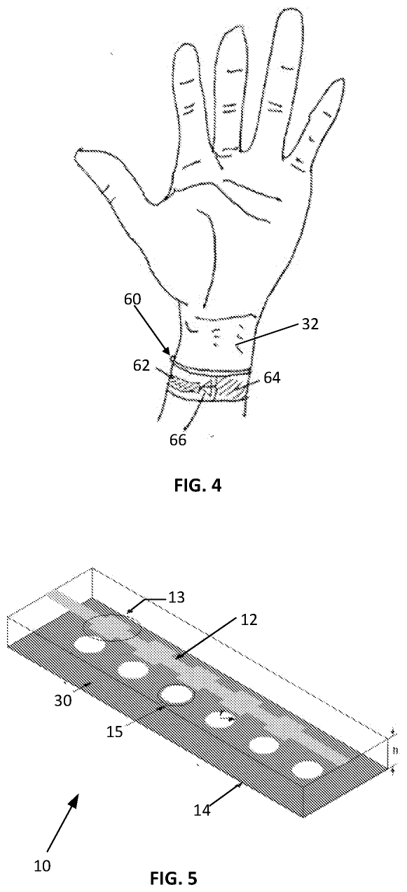

Some specific configurations of MLIN components are shown schematically in FIGS. 2 to 4.

In FIG. 2, a MLIN component 10 is in the form of a finger stall, and includes a microstrip conductor 12 spaced from a ground plane 14. The microstrip conductor 12 has a substantially smaller width than the ground plane 14. Microstrip conductor 12 has a first end 16 which is curved so as to hook over the end of the tip of a subject's finger 30 when worn, and a second end 18 which is substantially flat. Ground plane 14 is also curved, and is contoured to substantially accommodate the shape of the underside (i.e., the side opposite the nail) of the subject's fingertip. Alternatively, the ground plane 14 may be contoured to substantially accommodate the shape of the nail side of the subject's fingertip, with the microstrip 12 then extending along the underside of the subject's fingertip when the MLIN component 10 is worn. It may be more convenient for the microstrip 12 to be placed on the nail side of finger 30 as that may allow readier access to microstrip 12 for attaching signal lines as necessary.

In another configuration, shown in FIG. 3, a MLIN component is in the form of a ring 40. The ring 40 comprises a microstrip conductor 42 which, when the ring is worn, extends around the subject's finger 30. The microstrip 42 is opposite to, and spaced from, a ground plane 44, which likewise extends around finger 30. Microstrip 42 has a first end 46 to which an input signal can be applied, and a second end (not shown) which can be terminated by a load 20 as shown in FIG. 1.

In yet another configuration, shown in FIG. 4, a MLIN component is in the form of a bracelet 60. The bracelet 60 comprises a microstrip conductor 62 which, when the bracelet is worn, extends around the subject's wrist 32. The microstrip 62 is opposite to, and spaced from, a ground plane 64, which likewise extends around wrist 32. Microstrip 62 has a first end 66 to which an input signal can be applied, and a second end (not shown) which can be terminated by a load 20 as shown in FIG. 1.

In each of FIGS. 2, 3 and 4, only the conductors of the MLIN components 10, 40, 60 are shown for clarity. In practice, the conductors may be carried on a support structure, such as a rigid, semi-flexible or flexible support. For example, the support may be a polymer material to which the conductors are affixed, or into which they are embedded. In any event, the support structure improves user comfort and more readily allows the subject's body part (finger or wrist, for example) to be inserted between the microstrip and ground plane of MLIN component 10, 40 or 60 such that the body part acts substantially as the dielectric substrate for the MLIN component 10, 40, 60.

In use, an input signal is provided at an input port (such as at input end 46 of MLIN component 40), and a reflected signal is measured (for example, using signal input component 110 and/or processor 120). Because the subject's body part is contained within the MLIN component, it is subject to the main field of the MLIN component. Characteristics of the reflected signal can then be used to infer the glucose level in blood flowing through the subject's body part in a manner which will be described below in detail. In some embodiments, the transmitted signal, rather than the reflected signal, may equivalently be measured.

In some embodiments, it may be advantageous to modulate the structure of the microstrip conductor 12 (or 42 or 62) and/or of the ground plane 14 (or 44 or 64). For example, as shown in FIG. 5, which shows the MLIN component 10 in highly schematic form, the microstrip 12 may be patterned such that it has repeating units in the form of widened (e.g., rectangular) portions 13 at regular intervals. Alternatively, or in addition, the ground plane 14 may be patterned such that it has voids 15 at regular intervals. Patterning of the microstrip 12 and/or of the ground plane 14 improves the sensitivity of the glucose sensor apparatus 100, as it ensures that the input signal crosses the substrate more often, thus enhancing the interactions of the main field with the substrate 30. The centres of the widened portions 13 and voids 15 are preferably in register with each other to ensure optimal performance.

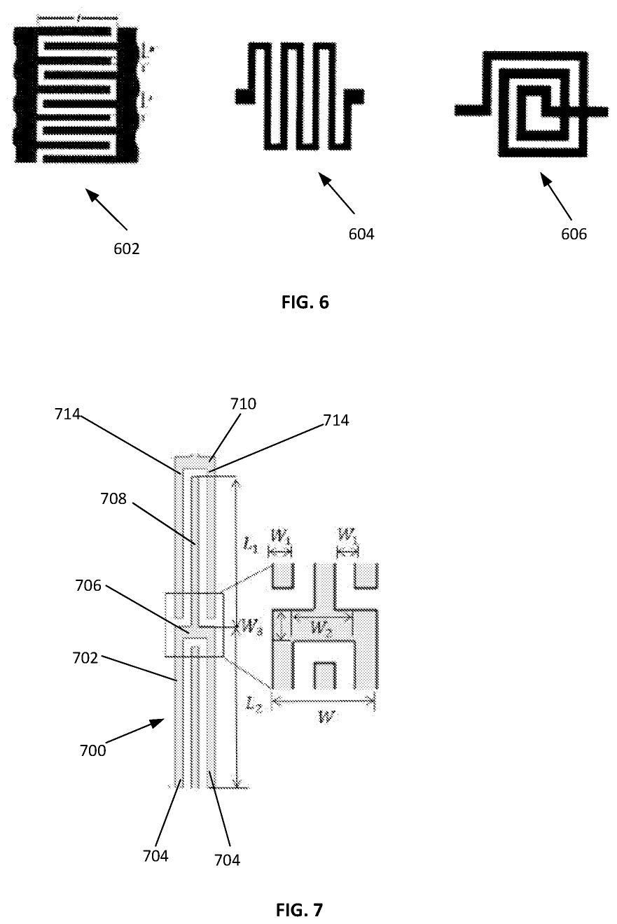

The patterning of the microstrip in FIG. 5 is in the form of plain squares or rectangles 13. It will be appreciated that other shapes are also possible. Some examples are shown in FIG. 6. For example, each unit 13 of the patterned microstrip 12 may be in the form of an interdigital capacitor 602, a meander inductor 604, or a spiral inductor 606.

One particularly advantageous form of patterned microstrip conductor is shown in FIG. 7, in which repeat units of a microstrip conductor 700 are in the form of T-shaped or Y-shaped elements 702. Each repeat unit 702 has a first pair of parallel legs 704 that are connected at a T-junction 706 to a third leg 708. The parallel legs 704 extend in one direction from the T-junction 706 and the third leg 708 extends in the opposite direction from the T-junction 706. To form the conductive structure 700, the third leg 708 is disposed between the parallel legs 714 of a like element 710, and this structure is repeated with additional T-shaped conductive elements (not shown).

Advantageously, when deployed in place of the microstrip 12 of MLIN component 10 of FIG. 2, the structure of microstrip conductor 700 may result in significantly higher penetration of the electric field into the substrate 30 between the microstrip conductor 700 and its corresponding ground plane 14. This may result in a sensitivity that is up to 10 times higher than that of the MLIN component 10. Some experimental tests of the microstrip conductor 700 are described below.

The particular examples shown in FIGS. 2, 3 and 4 are suitable for wearing by a subject for monitoring a glucose level of the subject. However, it will be appreciated that other configurations are possible. For example, a finger stall type device such as MLIN component 10 may be installed in a housing into which the subject may insert his or her finger such that it snugly fits within, and forms the dielectric substrate of, MLIN component 10. The MLIN component 10 may be supported within the housing in any suitable fashion. The housing may also contain the signal input component 110 and processor 120, such that the glucose monitoring apparatus is substantially self-contained.

The housing may itself be in the form of a finger stall, ring or bracelet so as to accommodate the microstrip conductor and ground plane in suitable fashion proximate an inner surface of the housing. For example, a microstrip conductor 42 and ground plane 44 of the MLIN component 40 shown in FIG. 3 may be embedded in, or attached to, an internal surface of a ring-shaped housing such that they contact the skin of a subject when worn by the subject. The ring-shaped housing may also contain a signal input component 110, a power source, and at least one processor, such as the processing device 120. In some embodiments, the ring-shaped housing may also contain a communications component for transmitting measured signals (for example, the raw reflected signal or the reflected signal with some preprocessing applied) to an external processing component for estimation of a glucose concentration based on the measured signals. The communications component may transmit and receive data wirelessly, for example via WiFi or Bluetooth, or via a wired connection to the external processing component. Similar considerations apply to the other configurations of MLIN component 10, 60 shown in FIGS. 2 and 4.

Embodiments of the present invention may include one or more of the following features:
Sensing the glucose level by using the main field, i.e., using the object under sensing as the substrate of a MLIN. Main field based glucose sensing is compared below to the fringing field approach adopted previously.
Using sensing parameters other than the magnitude of the reflection coefficient, for example the other components of the reflection coefficient, including the real part, imaginary part, and phase, and other parameters of the reflected signal, e.g. the input impedance.

### Testing of MLIN Components 10, 40, 60

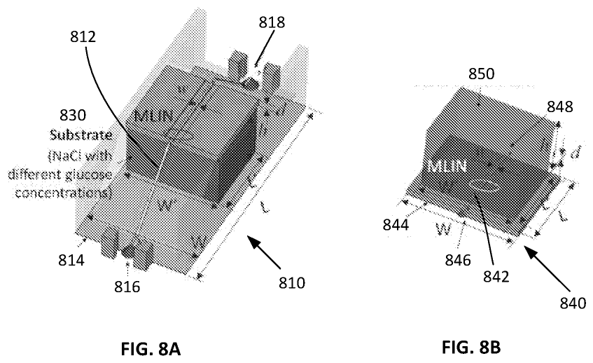

In order to compare the sensor of certain embodiments of the present invention to prior art sensors, a model was built and fabricated with the sensing target being in the shape of a block. The experimental model is depicted schematically in FIG. 8A. A comparison model, configured according to the existing MLIN-based solution that uses fringing fields, was also built and fabricated and is shown schematically in FIG. 8B. All the models were built using CST microwave studio of CST Computer Simulation Technology GmbH.

In FIG. 8A, the MLIN 812 runs on top of the block 830 (substrate) under sensing, a distance of d away from the block 830, and is bent to facilitate connections to SMA (SubMiniature version A) connectors at the two ends (input port 816 and output port 818). The ground plane 814 is at the back of the structure. This structure is called glucose-substrate MLIN (G-sub MLIN) in the below discussion of experimental results. In FIG. 8B, the block 850 under sensing is the same size as that in FIG. 8A. It is placed at a distance of d above the MLIN 842 (which is disposed opposite ground plane 844), which has an input port 846 and an output port 848, again terminated by SMA connectors. An FR4-grade material was used as the substrate of the MLIN. The configuration in FIG. 8B is referred to as glucose-fringe-field MLIN (G-FF MLIN) in the discussion below.

The structures in FIGS. 8A and 8B are two-port structures. In each case, Port 2 (the output port 818 or 848) is terminated with a load. The load can be open circuit, short circuit, matched, capacitive load, or inductive load. Sensing parameters that can be measured in the arrangements of FIGS. 8A and 8B are the reflection coefficient (S₁₁) and the input impedance (Z₁₁), including different components of these parameters, namely the real part, imaginary part, magnitude, and phase of each.

The sensitivity, s, is defined as follows:

$$s = \frac{Δ P}{Δ C} , \qquad ( 1 )$$

where P is the sensing parameter. P can be, for example, |S₁₁|, phase (S₁₁), Re(S₁₁), Im(S₁₁), |Z₁₁|, phase (Z₁₁), Re(Z₁₁), Im(Z₁₁). C is the glucose concentration.

Detailed dimensions of the G-sub MLIN are shown in FIG. 8A. The width of the MLIN 812 is w, the block 830 of solution under sensing has a size of L′×W′×h, and the ground plane 814 has a size (area) of W×L. The G-sub MLIN was fabricated with w=2 mm, d=0.2 mm, h=15 mm, W′=25 mm, L′=20 mm, W=30 mm, and L=65 mm. The material of the substrate of the MLIN is the block 830 under sensing. The height, h, is set to be 15 mm to mimic the thickness of a finger. The block 830 contains a solution for which the glucose level is to be sensed. The solution may be a 0.9% NaCl solution with different glucose concentrations.

The G-FF MLIN structure in FIG. 8B corresponds to a previously known type of capacitive fringing field-based MLIN sensor. It was fabricated with the width of the MLIN 842 set to be 2 mm. The substrate is an FR4-grade material with a dielectric constant of 4.1, a thickness (h) of 2 mm, a length of L=30 mm, and a width W=35 mm. A dielectric block 850 with the same size as that in the G-sub MLIN case (h=15 mm, L′=20 mm, W′=25 mm) was placed at a distance of d=0.2 mm above the MLIN. The material of this dielectric block 850 is the solution under sensing.

Experiments were conducted for studying the sensitivity of the structures to the change of glucose concentrations in blood. In this study, sodium chloride (NaCl) solutions (0.9%) at different glucose concentrations are used to mimic blood at different glucose levels, as this type of solution is known to have similar electromagnetic properties to blood. Seven different NaCl (0.9%) samples with respective concentrations of 5,000, 2,500, 1,250, 625, 312, 156, and 78 mg/dL were prepared. For the preparation of the samples, 0.9% NaCl solution (Baxter) and D-glucose (99.5%, Fluka) were used. A Rohde & Schwarz ZVH8 vector network analyzer was used for measuring S₁₁.

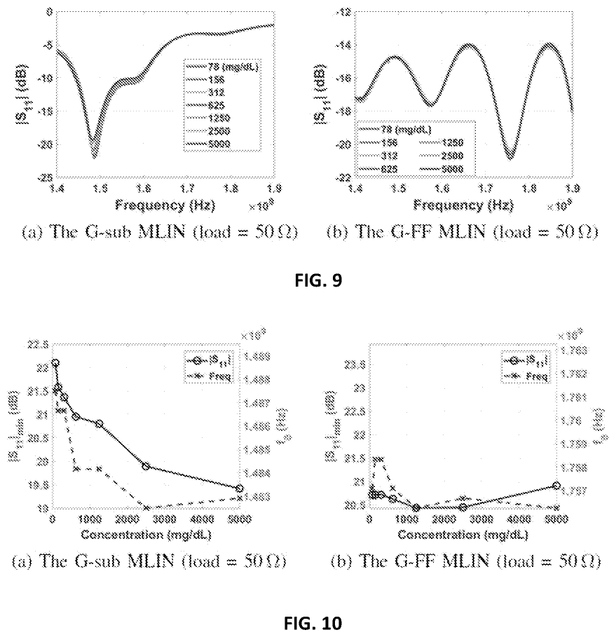

FIGS. 9(a) and 9(b) show the measured |S₁₁| of the G-sub MLIN 810 and that of the G-FF MLIN 840 versus frequency for NaCl at different glucose concentrations, respectively. The frequency range is 1.4 GHz to 1.9 GHz where the structure shows resonance. The load is 50Ω. As can be seen in FIGS. 9(a) and 9(b), the change in the concentration causes a change in the resonance of the structure in terms of the magnitude (|S₁₁|ₘᵢₙ) and the resonant frequency (A). In order to further examine the sensitivity, |S₁₁|ₘᵢₙ and f₀ versus the concentration were plotted for the G-sub MLIN 810 and G-FF MLIN 840 and shown in FIGS. 10(a) and 10(b), respectively. The range for the plot of |S₁₁|ₘᵢₙ is 3.5 dB and that for f₀ is 7 MHz.

It is shown clearly that the changes in both |S₁₁|ₘᵢₙ and f₀ for the G-sub MLIN 810 in FIG. 10(a) are much steeper than those for the G-FF MLIN 840 in FIG. 10(b). This indicates that the G-sub MLIN 810 has much higher sensitivity than the G-FF MLIN 840. This is owing to the fact that the object under sensing interacts with the main field of a MLIN in the Gsub MLIN 810. This is much stronger than the fringing field experienced by the object in the G-FF MLIN 840.

Additionally, the changes of both parameters of the G-sub MLIN 810 are monotonic as shown in FIG. 10(a) whereas for the GFF MLIN 840, as shown in FIG. 10(b), the change of |S₁₁|ₘᵢₙ with respect to the concentration is concave whereas the change of f₀ with respect to the concentration is undulating (rippled).

A monotonic variation of a measured parameter tends to provide high sensing accuracy due to less ambiguity. A concave or a rippled case is ambiguous for sensing. For the whole glucose concentration range of interest, ambiguous calibration curves are not preferred because they lead to low sensing accuracy.

The sensitivities (s) in terms of |S₁₁| for the curves in FIGS. 10(a) and 10(b) were calculated using Equation (1). The maximum, minimum, and average sensitivities (|s|ₘₐₓ, |s|ₘᵢₙ and |s|ₐᵥₑ) are shown in Table I.

TABLE I
SENSITIVITY IN TERMS OF |S₁₁| OF
G-SUB AND G-FF MLIN (dB/(mg/dL))

| | G-sub | G-FF |
|---|---|---|
| \|s\|ₘₐₓ | 6.60 × 10⁻³ | 3.12 × 10⁻⁴ |
| \|s\|ₘᵢₙ | 1.90 × 10⁻⁴ | 4.96 × 10⁻⁶ |
| \|s\|ₐᵥₑ | 1.80 × 10⁻³ | 1.38 × 10⁻⁴ |

As can be seen in Table I, all the values for the G-sub MLIN are at least 10 times higher than the corresponding sensitivities of the G-FF MLIN. Moreover, the sensitivity of the G-sub MLIN has an average value of 1.80×10⁻³ mg/(dL) which is about 10 times higher than one previously proposed patterned MLIN sensor (see V. Turgul and I. Kale, Sensors, 18665(R1), 1, 2017, which reported 2.21×10⁻⁴ mg/(dL) at low concentrations) and comparable with another previously proposed patterned MLIN sensor (see Harnsoongnoen et al, IEEE Sensors Journal 17.6 (2017):1635-1640, which reported 2×10⁻³ mg/(dL) at high concentrations). For both of these previously proposed MLIN-based sensors, fringing fields are used for sensing.

The reason for the significant increase in sensitivity of the G-sub MLIN is the location where the target under sensing is placed. In the G-sub MLIN, the target solution under sensing serves as the substrate of a MLIN, where the electromagnetic fields are highly confined, whereas in the G-FF MLIN case, the target solution only interacts with the fringing field of the MLIN which is much weaker than the main field. Fields in the substrate of the G-sub MLIN 810 are much more highly confined compared to those in the air (the fringing field), which is due to the location of the ground plane as well as a higher dielectric constant of the substrate compared to air. Therefore, when the target under sensing serves as a substrate between the signal line and the ground plane, the change of glucose concentration generates significant effects on the characteristics of the MLIN. Consequently, it can considerably change the parameters of the MLIN, such as the reflection coefficient (S₁₁), input impedance (Z₁₁), transmission coefficient (S₂₁), and characteristic impedance (Z₀), etc.

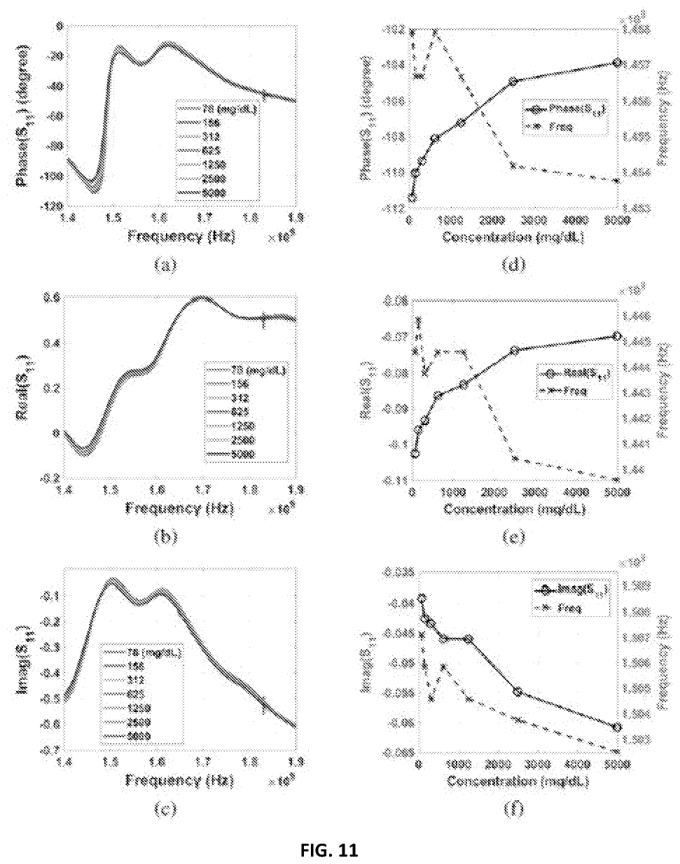

As shown in FIGS. 9 and 10, the G-sub MLIN structure 810 shows much higher sensitivity than the G-FF MLIN 840, in terms of |S₁₁|. We also investigated the sensitivity of the other components of S₁₁ for the G-sub MLIN 810. FIGS. 11(a)-(c) show the measured phase (S₁₁), Re(S₁₁), Im(S₁₁) versus frequency (1.4 GHz-1.9 GHz), and FIGS. 11(d)-(f) show the change of the maximum (max)/minimum (min) values of these parameters over the frequency band of interest versus the concentration and the corresponding frequencies. In the case that a parameter has both a maximum and a minimum over the frequency range (such as in FIG. 11(c)), the steeper of the two was chosen (i.e., the one having the largest magnitude for the second derivative). This provides for relatively higher sensitivity. As shown in FIGS. 11(d)-(f), the phase, real part, and the imaginary part of S₁₁ change monotonically with the change of concentration. In FIGS. 11(d)-(f), phase (S₁₁) is in the range of 10°, and Re(S₁₁) and Im(S₁₁) are in the range of 0.5 in ratio. Comparing these four components of S₁₁, it can be seen that they are all sensitive to the change of the glucose concentration and they are distinguishable from each other. In terms of the change in the frequencies at which the physical values (|S₁₁|, phase (S₁₁), Re(S₁₁), Im(S₁₁)) were recorded, they are all plotted with a range of 7 MHz. The curves are not monotonic except for |S₁₁|. As discussed, they are not all suitable for an accurate estimation of glucose concentration in the whole range of interest, but they can be suitable for the estimation in a small range locally.

The normalized input impedance (z₁₁, where z₁₁=Z₁₁/Z₀) can be either measured directly or calculated from the measured S₁₁. Equation (2) shows the relation between z₁₁ and S₁₁.

$${z}_{11} = \frac{1 + {S}_{11}}{1 - {S}_{11}} \qquad ( 2 )$$

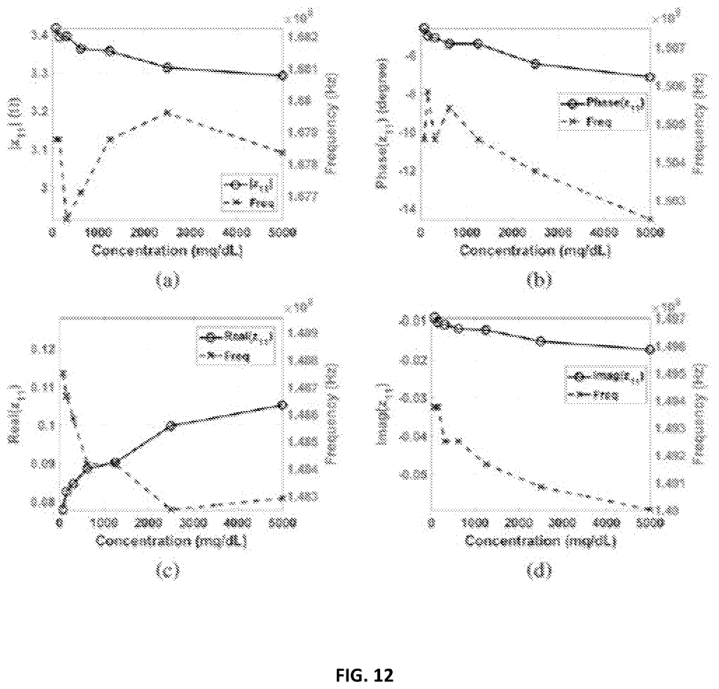

FIG. 12 shows the change of the max/min values of z₁₁, phase (z₁₁), Re(z₁₁), and Im(z₁₁) over the frequency band of 1.4 GHz-1.9 GHz versus the concentration. Again, in the cases when there are both max and min values, a steeper case was chosen. In each panel, the corresponding frequencies of the parameter values were also plotted. In FIG. 12, |z₁₁| is in the range of 0.5Ω, phase (z₁₁) is in the range of 10°, Re(z₁₁) and Im(z₁₁) are in the range of 0.5Ω. The changes in the four components of z₁₁ are all monotonic and independent of one another. The change in the recorded frequencies are plotted with the same range (7 MHz). As shown, only Re(z₁₁) and Im(z₁₁) show monotonic decreases.

Compared to the sensitivity of S₁₁ shown in FIG. 10(a) and FIGS. 11(d)-(f) and those of z₁₁ shown in FIG. 12, the sensitivities of both the physical values and the corresponding frequencies showed distinguishable trends and steepness, which indicates the possibility of the use of multi-variable crosschecking for sensing. As will be described in more detail below, algorithms can be developed to demonstrate the improvement in sensing accuracy when different sensing components from the same parameter or from different parameters of the same structure are used for crosschecking.

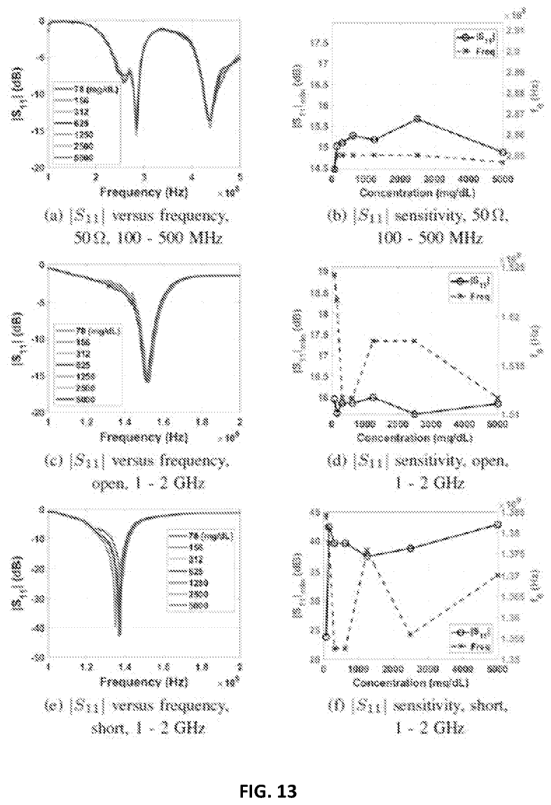

The sensitivity of the proposed MLIN configuration in a different frequency band, and that when the load is changed to open and short, were examined. FIGS. 13(a)-(b) show the measured magnitude of S₁₁ versus frequency and its sensitivity in the frequency band of 100 MHz to 500 MHz. This frequency range was chosen for the reason that it falls in the range where the molecules are known to interact with the waves (see A. Caduff et al, “First human experiments with a novel non-invasive, non-optical continuous glucose monitoring system”, Biosensors and Bioelectronics, 209-217, 2003). In FIG. 13(b), the vertical range of |S₁₁|ₘᵢₙ is 3.5 dB and the range of the frequency is 7 MHz, which is set to be the same as that in FIG. 10. Compared to the sensitivity of |S₁₁| in the frequency band of 1.4-1.9 GHz, the sensitivity of the same structure in the MHz range is considerably lower. Although Caduff et al discussed that a range at MHz would be sensitive because it includes low frequencies, the effect of β-dispersion and DC conductivity, and also avoids the high-frequency problems such as the electrode polarization and huge signals from the α-dispersion in tissues, the best sensing frequency range for embodiments of the present invention is actually in a high frequency range as a result of the structure of the sensing device, in which the object under sensing forms the substrate of the MLIN.

FIGS. 13 (c) and (d) show the measured |S₁₁| versus frequency and the |S₁₁| sensitivity when the load 20 is open. The frequency range is slightly widened to be 1-2 GHz in order to capture the resonance. The range of |S₁₁|ₘᵢₙ in FIG. 13(d) is set to be 3.5 dB, the same as that of FIG. 10 for ease of comparison. The range of frequency is 15 MHz to capture the changes. Comparing FIG. 13(d) to FIG. 10(a), the sensitivity in terms of |S₁₁|ₘᵢₙ drops considerably when the load is changed from 50Ω to open. On the other hand, in the case of an open load, a bigger shift of the resonant frequency is introduced by the change of concentration, which is shown in FIG. 13(d). The results when the load 20 is changed to short are shown in FIGS. 13(e) and 13(f). The frequency is set to be 1-2 GHz to capture the resonance. In FIG. 13(f), the range of |S₁₁|ₘᵢₙ is set to be 25 dB and the range of f₀ is set to be 35 MHz to include the changes. As can be seen, the ranges of both changes are much bigger than those attained by previously proposed arrangements. However, the trend is not monotonic.

In FIG. 13, it can be seen that when either the frequency range or the load is changed, the sensitivity of the G-sub structure changes dramatically. Comparing FIGS. 13(b), 13(d), and 13(f) to FIG. 10(a), the same parameter in different situations shows quite different glucose concentration dependence. Accordingly, improvements to sensitivity may be obtained through crosschecking multiple parameters and multiple components of the parameters. An example of crosschecking using data from both frequency ranges when the load is 50Ω will be discussed in more detail below.

In order to investigate the effect on sensitivity of the use of multiple parameters and/or parameter components, algorithms for univariate estimation (estimation using a single component of a certain parameter), and multivariate estimation (estimation using multiple components of a parameter or multiple parameters) were proposed and tested. The data sets used for the estimation of glucose concentration were collected from the experiments on the G-sub 810 and G-FF 840 structures in FIGS. 8A and 8B for different parameters of the same setup (the same load and the same frequency range) and for different parameters of different setups (different loads and different frequency ranges).

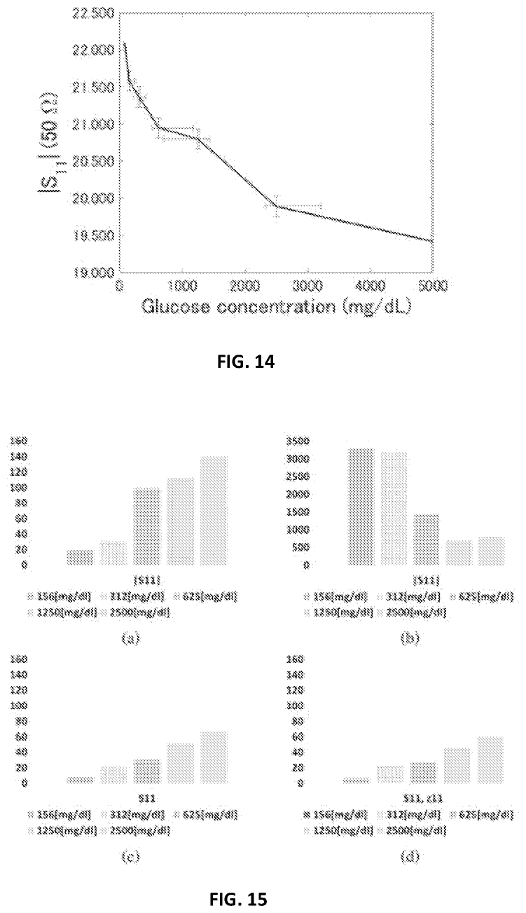

For the test, a pseudo-test-sample generation algorithm was implemented to generate the test sample denoted by Vₚ_{ᵢₕ}_{-Δf}_{ⱼ}_{-c}_{ₖ} where pᵢₕ represents the h^{th} component of the i^{th} MLIN parameter, Δfⱼ represents the j^{th} frequency range, and cₖ represents the k^{th} concentration. FIG. 14 illustrates the test sample generation process based on |S₁₁| when the load is 50Ω in the frequency range of 1.4-1.9 GHz. For each glucose concentration under inspection, cₖ, the algorithm generated test samples with a value of |S₁₁| within a deviation, which is 5% of the difference between the maximum and the minimum of the value of |S₁₁| at that concentration, indicated by the vertical error bars in FIG. 14. Details of this algorithm are included below.

Depending on the number of the components of a MLIN parameter, the MLIN parameters, and frequency range used for the estimation, the algorithms for glucose concentration estimation can be classified as follows.

Algorithm 1: Univariate or single-variate estimation (SV) for a single component of a single parameter, single frequency range (SCSP-SF)

Algorithm 2: Multi-variate estimation (MV) for the following situations:
Multiple components of a single parameter, single frequency range (MCSP-SF)
Multiple components of multiple parameters, single frequency range (MCMP-SF)
Multiple components of a single parameter, multiple frequency ranges (MCSP-MF)
Multiple components of multiple parameters, multiple frequency ranges (MCMP-MF)

Algorithm 3: Multi-variate estimation with Bin Correction (MV-BC), the meaning of and necessity for which is explained below.

For SV, the estimation is made by matching a test sample, Vₚ_{ᵢₕ}_{-Δf}_{ⱼ}_{-c}_{ₖ}, with a single-parameter data set collected from the experiment at one frequency range. FIG. 14 shows one example of a calibration curve that uses |S₁₁| when the load is 50Ω (frequency range 1.4-1.9 GHz).

The relationship between |S₁₁| and concentration is monotonic in this case. The horizontal error bars show the maximum likely concentration estimation error due to the perturbation induced, which corresponds to the vertical bars.

For MV, for example in the case of MCSP-SF, for a single parameter at a single frequency, different components (e.g. the real part, imaginary part, magnitude, and phase of a parameter) are used for the estimation of glucose concentration. The line segment (bin) connecting the two adjacent concentration points (e.g., from 156 mg/dL to 312 mg/dL) with the largest gradient among all the variables was used to calculate the glucose concentration. Note, the gradient of line segment for each component pᵢₕ, was standardized with the parameter values corresponding to the smallest concentration value of that component pᵢₕ.

The cases of MCMP-SF, MCSP-MF, and MCMP-MF are similar to that of MCSP-SF. For MCMP-SF, for the frequency range, Δfⱼ, measured data which contain multiple variables of multiple parameters are used for estimation. For MCSP-MF, for each specific pᵢₕ, the data corresponding to multiple frequencies are used to estimate the glucose concentration. For MCMP-MF, instead of using the data sets from only one single MLIN parameter in MCSP-MF, the exploration of maximal gradient, and concentration value matching is done for all MLIN parameters specified. For the sensitivity curves used for estimating glucose concentration, although it is monotonic, as shown in FIG. 14, it is possible that, by perturbation, the line segment chosen for calculation of glucose concentration is different from the expected one. In this situation, bin correction is proposed as follows.

Assuming that the deviation (i.e. the maximum and minimum of the data set of the MLIN parameter) and frequency, the ratio to calculate the deviation (i.e. 5% etc.), and (Maxₚ_{ᵢₕ}_{-Δf}_{ⱼ}−Minₚ_{ᵢₕ}_{-Δf}_{ⱼ}) are known, for each test sample point, the positive deviation and the negative deviation are used to calculate an expected left estimation error, and an expected right estimation error. Then the bin for final glucose concentration matching will be determined in a competitive way, that is, the bin with a smaller sum of expected errors is chosen. The detailed algorithm is included below. The error was calculated by summing up the difference between the estimated concentration and the actual one in the model.

5000 samples were generated using the pseudo-test-sample generation algorithm. The single-variate and multiple-variate algorithms proposed were applied to estimate the glucose concentration. FIG. 15 shows the error of estimations of glucose concentration based on the measured S₁₁ and z₁₁ of the Gsub MLIN as well as that of the G-FF MLIN for comparison. The load was 50Ω and the frequency range was 1.4-1.9 GHz. The bars in different colors show the estimation errors for different concentrations. FIGS. 15(a) and 15(b) show the estimated error based on single-variate of single parameter (|S₁₁|) at a single frequency range (SVSP-SF) for the G-sub MLIN and the G-FF MLIN, respectively. The vertical scale of FIG. 15(a) is 0-160 and that of FIG. 15(b) is 0-3500. Comparing FIGS. 15(a) and 15(b), the G-sub structure 810 has much higher estimation accuracy compared to the G-FF structure 840, which is due to the higher sensitivity of the G-sub structure 810 when the object under sensing serves as the substrate of a MLIN. This, again, successfully shows the higher sensitivity of the proposed MLIN configuration for glucose sensing. Moreover, as can be seen in FIG. 15(a), the G-sub structure shows higher accuracy at low glucose concentrations compared to that at high concentrations whereas the G-FF structure is the other way around.

FIGS. 15 (c) and 15(d) show the estimated error based on multivariate of a single parameter (the real part, imaginary part, magnitude, phase of the S₁₁) at single-frequency (MVSP-SF), and multi-variate of multiple parameters (S₁₁ and z₁₁) in a single frequency range (MVMP-SF) of the G-sub MLIN with 50Ω at the load, respectively. Comparing FIGS. 15(a) and 15(c), when multiple components of a single parameter were used for the estimation, the accuracy improved significantly. The accuracy further improved when multiple parameters were used, which is shown in FIG. 15(d).

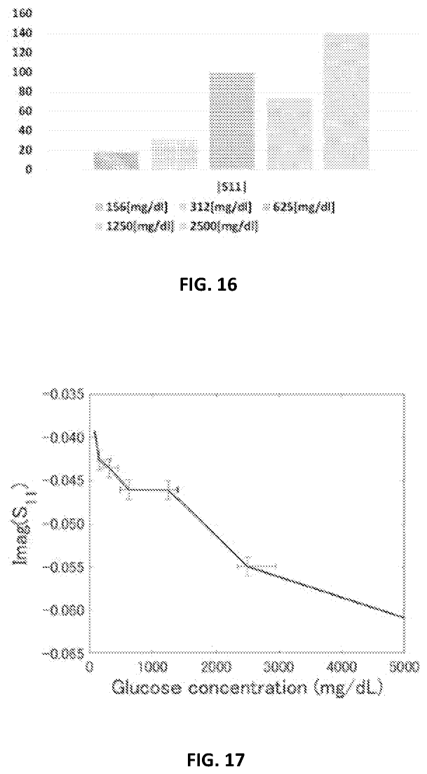

Besides the methods for a single frequency range, the method for multiple frequency ranges was tested. FIG. 16 shows the estimated concentration error when the measured |S₁₁| at the frequency ranges of 1.4-1.9 GHz and 100-500 MHz were used. Comparing FIG. 16 to FIG. 15(a), it is clear that adding data from another frequency range as additional reference data helps to increase the accuracy at certain concentrations. It is observed that the improvement is not significant, which is due to the low sensitivity of the structure under test at the additional frequency range (see FIG. 13(b)).

Accordingly, as can be seen from the above-discussed experimental results:
By having the object under sensing serve as the substrate of a MLIN, much higher sensitivity in terms of |S₁₁| is achieved. For example, an average sensitivity of 1.8×10⁻³ dB/(mg/dL) can be achieved, which is 10 times higher than the G-FF structure 840.
The sensitivity of the G-sub structure 810 can be enhanced by using multiple parameters and/or multiple parameter components. Each of the components of S₁₁ and z₁₁, for example, shows a distinguishable trend as a function of glucose concentration, thus facilitating crosschecking of inferred glucose concentration. Moreover, the sensitivity at different frequency bands, and with different loads versus the concentration is shown to be independent, which can be useful for crosschecking as well. These findings are important because they show that sensitivity can be increased without adding further sensor elements, which would introduce additional sources of error, additional interference, and require additional circuit space.

In the experimental study described above, a configuration 810 with an unpatterned MLIN and a perfect ground plane was studied, mainly to aid comparison to its fringing field counterpart 840. However, as discussed above, the sensitivity can be significantly enhanced by introducing patterns to the MLIN and/or to the ground plane such that interactions with electromagnetic waves can be enhanced by the pattern structures.

The device of certain embodiments of the invention is non-invasive and can be wearable. Thus it supports continuous monitoring. As mentioned previously and shown in FIGS. 2 and 3, the object under sensing can be a finger where glucose concentration level may vary. The signal input can be introduced at the tip of the finger 16 while at the other end 18 of the MLIN, different loads can be introduced. For example, an open circuit can be the load. One or more of the proposed configurations can fit in, for example, a finger stall to be a wearable device for continuous monitoring.

Testing of MLIN Component with Patterned Microstrip 700

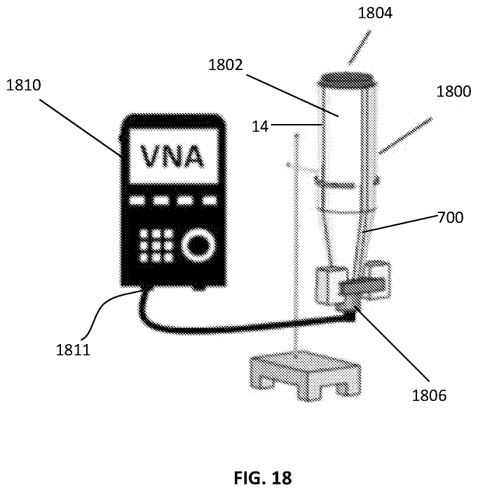

Referring to FIG. 18, the sensitivity of a proposed sensor 1800 using the microstrip 700 was tested using 0.9% NaCl aqueous (B. Braun Medical Industries) with different glucose concentrations (D-glucose, C₆H₁₂O₆, Sigma-Aldrich). The solution is contained in a 0.6 mL graduated microtube 1804 (Scientific Specialties, Inc. (SSI), USA) for measurements.

A test sensor 1800 was built by fabricating a housing structure 1802 by 3D printing. The housing structure 1802 houses the microtube 1804 with NaCl as the substrate and supports the layouts of the signal line 700 and the ground plane 14. For the housing 1802, the thickness of the wall is 1.5 mm, the total height is 31 mm (11 mm for the cone and 20 mm for the cylinder), and the material is HP 3D High Reusability PA 12 (εᵣ≈4.4, σ≈0 S/m, certified for medical devices). Two slits are introduced to the cylinder to provide tolerance to a variation of the size of the tube 1804. The signal line and ground plane were copper (1 oz) fabricated using PCB etching on a thin flexible film (polyimide, εᵣ≈3.4, σ≈0 S/m, 0.1 mm in thickness) separately. They were cut and pasted on the 3D printed housing. The dimensions of the T-shaped pattern (see FIG. 7) are, W₁=0.11 mm, W₂=0.32 mm, W₃=0.17 mm, L₁=2.1 mm, and L₂=2.3 mm. The width of microstrip line for both the MLIN structures is W=0.57 mm. The signal input is introduced from the tip of the tube through a SubMiniature (SMA) connector 1806. A holder for the SMA connector is included in the housing for accurate positioning between the connector, the signal line, and the ground plane, and for robustness of the sensor. The other end of the sensor is an open cylinder which allows an insertion of a tube. Different loads can be introduced between the signal line and the ground along the periphery of the cylinder. In this study, an open circuit was chosen. Another sensor without the patterns in the MLIN was fabricated and measured for comparison.

A total of twelve samples were prepared to test the sensitivity of the sensor. Each sample was prepared with exact ratios of 0.9% NaCl aqueous and D-glucose powder at different glucose concentrations. The samples are separated into two groups. One has low concentrations ranging from 0-120 mg/dL with a step of 20 mg/dL. The other one has high concentrations ranging from 100-600 mg/dL with a step of 100 mg/dL.

The SMA connector 1806 was connected to Port 1 (1811) of a vector network analyzer 1810 (VNA, Keysight N52498). The measurements were conducted five times and the results were averaged for further analyses. The change of |S₁₁| over the corresponding change of glucose concentrations (denoted as C) was used as a sensing parameter, S=Δ|S₁₁|/ΔC, for evaluating the sensitivity of the sensor 1800.

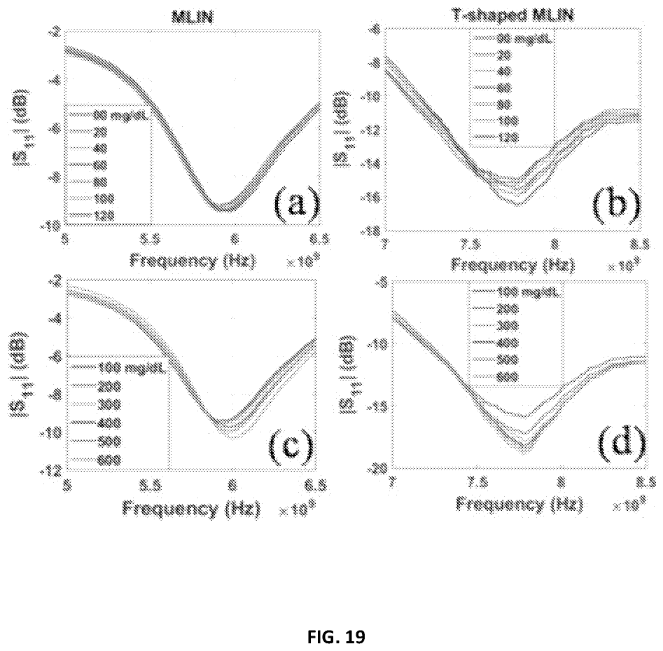

FIGS. 19(a) and 19(b) show the average |S₁₁| versus frequency for the proposed sensor 1800 having patterned microstrip 700 and that of the MLIN sensor (without a pattern in the MLIN) at low concentrations, respectively. The resonant frequencies are at 7.8 GHz and 6 GHz, respectively. The average quality factors (Q-factor) are 9 and 15 for the MLIN and the proposed structure 1800, respectively. The bandwidths are much wider compared to a resonator. It is seen that the pattern in the MLIN shifts the resonance higher. In both cases, it is observed that the minimum |S₁₁| decreases with an increasing glucose concentration. Moreover, the resonant frequency is shifted higher when the concentration increases. FIGS. 19(c) and 19(d) show the measurements at high concentrations. The same trends in terms of resonant frequency and |S₁₁|ₘᵢₙ are observed for both structures.

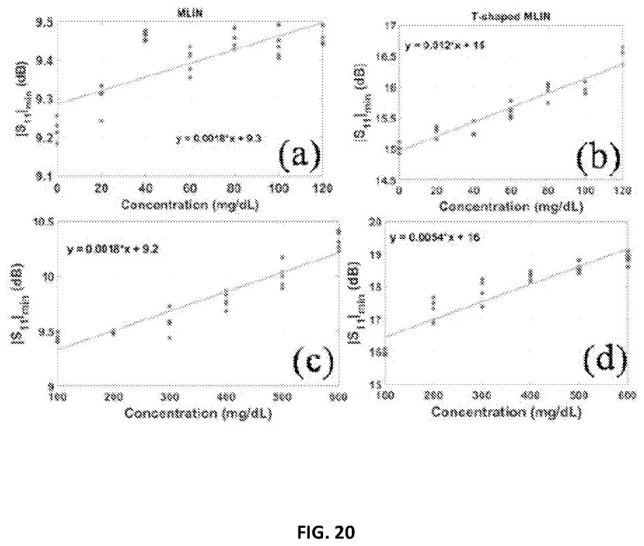

FIG. 20 shows the recorded |S₁₁|ₘᵢₙ at each concentration. Linear regression was applied to the data. The slope of the curve indicates the sensitivity of the structure in dB/(mg/dL). The first row in FIG. 20 shows the results of the MLIN (left) with no patterning, and the proposed structure 1800 (right) at low concentrations. The unpatterned MLIN and the T-shaped pattern MLIN produce slopes of 1.8×10⁻³ dB/(mg/dL) and 1.2×10⁻² dB/(mg/dL), respectively. This implies that the proposed structure 1800 with patterned microstrip 700 is about 10 times more sensitive compared to the unpatterned MLIN 12. At high concentrations, the proposed structure 1800 shows a slope of 5.4×10⁻³ dB/(mg/dL) which is three times of that of the unpatterned MLIN structure (1.8×10⁻³ dB/(mg/dL)). The proposed structure 1800 shows much higher sensitivity, especially at low glucose concentrations, compared to an unpatterned MLIN of the same sensing configuration.

Compared to a MLIN without any pattern, the proposed MLIN shows much higher sensitivity, about 10 times more at low glucose concentrations and 3 times higher at high concentrations. This sensitivity is much higher than that of the state-of-the-art MLIN-based sensors for the same concentrations and is comparable to resonance-based microstrip sensors with improved robustness, i.e. a wider band and significant mitigation of the error sources from pressure and positioning.

### Pseudo-Test-Sample Generation Algorithm for Generating the Test Samples

For the test, a pseudo-test-sample generation algorithm was implemented. Suppose that the data sets can be denoted using Vₚ_{ᵢₕ}_{-Δf}_{ⱼ}_{-c}_{ₖ} where pᵢₕ represents the h^{th} component of the i^{th} MLIN parameter, Δfⱼ represents the j^{th} frequency range, and cₖ represents the k^{th} concentration. For each Δfⱼ range, find Maxₚ_{ᵢₕ}_{-Δf}_{ⱼ}=max(Vₚ_{ᵢₕ}_{-Δf}_{ⱼ}_{-c}_{ₖ}|k=1), and Minₚ_{ᵢₕ}_{-Δf}_{_{j=min(V}}ₚ_{ᵢₕ}_{-Δf}_{ⱼ}_{-c}_{ₖ}|k=1). For each Vₚ_{ᵢₕ}_{-Δf}_{ⱼ}_{-c}_{ₖ}|k=1, generate a number of random test values (perturbation) RV with a given deviation value. The probability density of the perturbation RV is assumed to be Gaussian. The effect of the probability density function is to be investigated through the comparison between Gaussian and white noise function.

σₚ_{ᵢₕ}_{-Δf}_{ⱼ}=r(Maxₚ_{ᵢₕ}_{-Δf}_{ⱼ}−Minₚ_{ᵢₕ}_{-Δf}_{ⱼ}) (3)

where r is a ratio to the difference between the maximum and minimum of the data set. For each concentration under investigation, the test sample is

S=Vₚ_{ᵢₕ}_{-Δf}_{ⱼ}_{-c}_{ₖ}+RV(Vₚ_{ᵢₕ}_{-Δf}_{ⱼ}_{-c}_{ₖ},σₚ_{ᵢₕ}_{-Δf}_{ⱼ}) (4)

FIG. 17 shows one example of the test sample generated based on the measured imaginary part of S₁₁ when the load is 50Ω in the range of 1.4-1.9 GHz. The horizontal axis is the glucose concentration in mg/dL. In FIG. 17, the vertical error bars indicate the deviation at the same glucose concentration, and the horizontal error bars indicate corresponding concentration estimation error due to the perturbation induced.

### Algorithm for Bin Correction

Given (Maxₚ_{ᵢₕ}_{-Δf}_{ⱼ}−Minₚ_{ᵢₕ}_{-Δf}_{ⱼ}) for a specific parameter pᵢₕ, and a frequency range Δfⱼ, a ratio to the difference between the maximum and minimum of the data set (r, e.g., 5%), and the simulated data sets denoted using Vₚ_{ᵢₕ}_{-Δf}_{ⱼ}_{-c}_{ₖ}:

1) A multiple varying test sample Sₚ_{ᵢₕ}_{-Δf}_{ⱼ}_{-c}_{ₖ}^{MV} is a test sample vector consisting of the components for different parameters and different frequencies at a specific concentration C.
2) For each component of a multiple variate test sample, Sₚ_{ᵢₕ}_{-Δf}_{ⱼ}_{-c}_{ₖ}^{MV}, expand it to a pair as follows: [Sₚ_{ᵢₕ}_{-Δf}_{ⱼ}_{-c}_{ₖ}^{MV}−r(Maxₚ_{ᵢₕ}_{-Δf}_{ⱼ}−Minₚ_{ᵢₕ}_{-Δf}_{ⱼ})] and [Sₚ_{ᵢₕ}_{-Δf}_{ⱼ}_{-c}_{ₖ}^{MV}+r(Maxₚ_{ᵢₕ}_{-Δf}_{ⱼ}−Minₚ_{ᵢₕ}_{-Δf}_{ⱼ})].
3) This pair is used to look up the model points to get an expected left estimation error, e_{L} and an expected right estimation error, e_{R}. The errors are summed to obtain a total expected estimation error, eₜ=e_{L}+e_{R}. It is clear that the larger the value of eₜ, the lower the reliability of the estimation.
4) Calculate all the e_{L} and e_{R} for all components from Sₚ_{ᵢₕ}_{-Δf}_{ⱼ}_{-c}_{ₖ}^{MV} and the maximum of e_{L} and e_{R} is summed up to obtain a sum expected estimation error e_{S}.
5) Use the bin with the smallest e_{S} from multiple frequencies of a single parameter, or multiple frequencies of multiple parameters for the final estimation of the glucose concentration.

### Processor Device 120

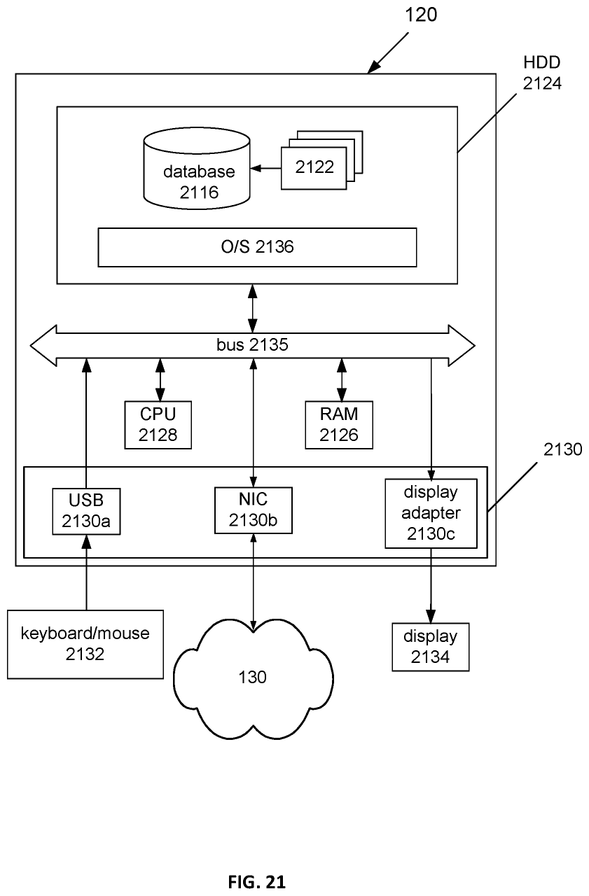

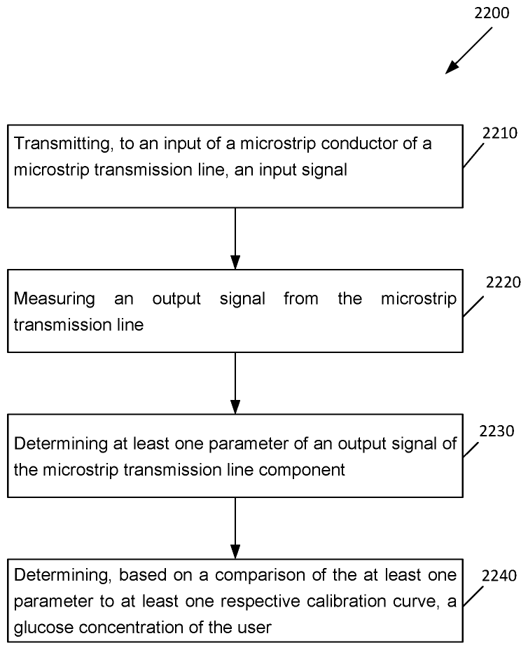

Turning now to FIG. 21, an exemplary architecture of a processor device 120 is shown. As discussed above, the processor device 120 is, or comprises, a concentration determining component that receives raw or pre-processed output signals (such as reflected signals measured at the input 16, responsive to an input signal provided by the signal input component 110) from the MLIN component 10, compares one or more parameters derived from the output signals to one or more corresponding calibration curves, and determines, from the comparison, an estimated glucose concentration.

In this example, the processor device 120 is a server computing system. In some embodiments, the server 120 may comprise multiple servers in communication with each other over a communications link 130, for example over a local area network or a wide-area network such as the Internet. The server 120 may communicate with other components of the glucose monitoring apparatus 100 (typically, the signal input 110 and/or another processing device that is in communication with the signal input 110) over the communications link 130 using standard communication protocols, for example a wireless communication protocol.

The components of the server 120 can be configured in a variety of ways. The components can be implemented entirely by software to be executed on standard computer server hardware, which may comprise one hardware unit or different computer hardware units distributed over various locations, some of which may require the communications network 130 for communication. A number of the components or parts thereof may also be implemented by application specific integrated circuits (ASICs) or field programmable gate arrays.

In the example shown in FIG. 21, the server 120 is a commercially available server computer system based on a 32 bit or a 64 bit Intel architecture, and the processes and/or methods executed or performed by the server 120 are implemented in the form of programming instructions of one or more software components or modules 2122 stored on non-volatile (e.g., hard disk) computer-readable storage 2124 associated with the server 120. At least parts of the software modules 2122 could alternatively be implemented as one or more dedicated hardware components, such as application-specific integrated circuits (ASICs) and/or field programmable gate arrays (FPGAs).

The server 120 comprises one or more of the following standard, commercially available, computer components, all interconnected by a bus 2135:

(a) random access memory (RAM) 2126;
(b) at least one computer processor 2128, and
(c) external computer interfaces 2130:
(i) universal serial bus (USB) interfaces 2130a (at least one of which is connected to one or more user-interface devices, such as a keyboard, a pointing device (e.g., a mouse 2132 or touchpad),
(ii) a network interface connector (NIC) 2130b which connects the computer system 120 to a data communications network 130; and
(iii) a display adapter 2130c, which is connected to a display device 2134 such as a liquid-crystal display (LCD) panel device.

The server 120 may comprise a plurality of standard software modules, including an operating system (OS) 2136 (e.g., Linux or Microsoft Windows).

Advantageously, the database 2116 forms part of the computer readable data storage 2124. Alternatively, the database 2116 is located remote from the server 120 shown in FIG. 21. The database 2116 may store data for use by software modules 2122 to execute particular functions. For example, calibration curves such as those shown in FIGS. 10-14, 17 and 20 may be stored in the database 2116.

The boundaries between the modules and components in the software modules 1622 are examples only, and alternative embodiments may merge modules or impose an alternative decomposition of functionality of modules. For example, the modules discussed herein may be decomposed into submodules to be executed as multiple computer processes, and, optionally, on multiple computers. Moreover, alternative embodiments may combine multiple instances of a particular module or submodule. Furthermore, the operations may be combined or the functionality of the operations may be distributed in additional operations in accordance with the invention. Alternatively, such actions may be embodied in the structure of circuitry that implements such functionality, such as the micro-code of a complex instruction set computer (CISC), firmware programmed into programmable or erasable/programmable devices, the configuration of a field-programmable gate array (FPGA), the design of a gate array or full-custom application-specific integrated circuit (ASIC), or the like.

Each of the blocks of the flow diagrams of the processes of the server 120 (for example, process 2200 shown in FIG. 22) may be executed by a module (of software modules 2122) or a portion of a module. The processes may be embodied in a non-transient machine-readable and/or computer-readable medium for configuring a computer system to execute the method. The software modules may be stored within and/or transmitted to a computer system memory to configure the computer system to perform the functions of the module.

The server 120 normally processes information according to a program (a list of internally stored instructions such as a particular application program and/or an operating system) and produces resultant output information via input/output (I/O) devices 2130. A computer process typically comprises an executing (running) program or portion of a program, current program values and state information, and the resources used by the operating system to manage the execution of the process. A parent process may spawn other, child processes to help perform the overall functionality of the parent process. Because the parent process specifically spawns the child processes to perform a portion of the overall functionality of the parent process, the functions performed by child processes (and grandchild processes, etc.) may sometimes be described as being performed by the parent process.

The software modules 2122 of server 120 may comprise the concentration determining component, as discussed above. Software modules 2122 may also comprise a control module for causing the signal input component 110 to transmit an input signal to the input 16 of MLIN component 10. The control module may be configured to cause the signal input component 110 to transmit input signals of varying frequency, for example. In some embodiments, the control module may request the signal input component 110 to transmit input signals to the MLIN component 10 at regular intervals, in order to substantially continuously monitor the glucose level of a subject who is in contact with (e.g., by wearing) MLIN component 10.

Although depicted as a separate server computing system 120 in FIGS. 1 and 21, it will be appreciated that some or all of the functionality of the processing device 120 may be implemented in hardware components that are contained in a housing of a substantially self-contained device. For example, if MLIN component 10 and signal input component 110 are contained in a finger stall-shaped, ring-shaped or bracelet-shaped housing as described above, then the concentration determining component may have similar architecture to server 120, but with certain hardware components such as USB 2130a and keyboard/mouse 2132 being omitted in order to aid miniaturisation into a wearable device. Alternatively, the concentration determining component may comprise software instructions that are stored on memory of, and executable by a processor of, the wearable device.

Turning to FIG. 22, there is shown a flowchart of a method 2200 for monitoring blood glucose concentration in a subject. One or more of the blocks of the flowchart of FIG. 22 may be implemented by the signal input component 110 and/or the processing device 120 (such as server 120 of FIG. 21).

The method 2200 comprises a first operation 2210 of transmitting, to an input of a microstrip conductor, an input signal. As described above, the microstrip conductor (such as microstrip conductor 12, 42 or 62) is arranged relative to a ground plane (e.g., 14, 44 or 64) to define a space to receive a body part of the subject, such as a finger or wrist of the subject. The microstrip conductor and the ground plane together function as a microstrip transmission line, and the dielectric substrate of the microstrip transmission line is the body part of the subject.

Next, an operation 2220 of measuring an output signal from the microstrip transmission line is performed. The output signal may be the reflected signal measured at the input port of the microstrip transmission line, for example.

At 2230, an operation of determining at least one parameter of the output signal of the microstrip transmission line component is performed. For example, this operation may be performed by the concentration determining component (e.g., server 120 or a software or hardware module of server 120). In some embodiments, the at least one parameter may be a reflection coefficient, an input impedance, or another parameter derived from one or both of those parameters. The at least one parameter may be a real or imaginary part, a phase, or a magnitude of the reflection coefficient or the input impedance.

At 2240, an operation of determining, based on a comparison of the at least one parameter to at least one respective calibration curve, a glucose concentration of the user is performed. This operation is performed by the concentration determining component (e.g., server 120 or a software or hardware module of server 120). For example, if the parameter is the imaginary part of the reflection coefficient, then the value of Im(S₁₁) may be used to read off the corresponding glucose concentration from the calibration curve shown in FIG. 17, or another, similar calibration curve generated by means other than that described above.

Throughout this specification, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference to any prior art in this specification is not, and should not be taken as, an acknowledgment or any form of suggestion that the prior art forms part of the common general knowledge.

1. A non-invasive glucose monitoring apparatus, comprising:
at least one microstrip transmission line component comprising a microstrip conductor that is arranged relative to a ground plane such that a body part of a user is receivable in a space defined between the microstrip conductor and the ground plane, the microstrip transmission line component having an input port;
a signal input component for transmitting an input signal to the input port; and
a concentration determining component configured to:
determine at least one parameter of an output signal of the microstrip transmission line component; and
determine, based on a comparison of the at least one parameter to at least one respective calibration curve, a glucose concentration of the user.

2. A non-invasive glucose monitoring device according to claim 1, wherein the microstrip conductor is patterned.

3. A non-invasive glucose monitoring apparatus according to claim 2, wherein a pattern of the microstrip conductor comprises a plurality of repeating units spaced at regular intervals.

4. A non-invasive glucose monitoring apparatus according to claim 3, wherein individual units are one or more of: a rectangular element; an interdigitated capacitor; a meander inductor; or a spiral inductor.

5. A non-invasive glucose monitoring apparatus according to any one of claims 1 to 4, wherein the ground plane is patterned.

6. A non-invasive glucose monitoring apparatus according to any one of claims 1 to 5, wherein the at least one wearable transmission line component is in the form of a ring, a finger stall, a bracelet and/or an anklet.

7. A non-invasive glucose monitoring apparatus according to any one of claims 1 to 6, wherein an output port of the microstrip transmission line component is terminated via a load.

8. A non-invasive glucose monitoring apparatus according to claim 7, wherein the load is an open circuit, a short circuit, an impedance-matched load, a capacitive load or an inductive load.

9. A non-invasive glucose monitoring apparatus according to any one of claims 1 to 8, wherein the at least one parameter comprises at least one parameter derived from the input impedance and/or the reflection coefficient.

10. A non-invasive glucose monitoring apparatus according to claim 9, wherein the at least one parameter comprises one or more of: the real part of the input impedance, the imaginary part of the input impedance, the magnitude of the input impedance, the phase of the input impedance, the real part of the reflection coefficient, the imaginary part of the reflection coefficient, the magnitude of the reflection coefficient, and the phase of the reflection coefficient.

11. A non-invasive glucose monitoring apparatus according to any one of claims 1 to 10, wherein the concentration determining component is configured to determine the glucose concentration based on a plurality of parameters derived from the output signal.

12. A non-invasive glucose monitoring apparatus according to any one of the preceding claims, wherein the microstrip transmission line component is supported within a housing.

13. A non-invasive glucose monitoring apparatus according to claim 12, wherein the signal input component is within, extends from, or is attached to the housing.

14. A non-invasive glucose monitoring apparatus according to any one of the preceding claims, wherein the concentration determining component is in the form of computer-readable instructions stored on non-volatile storage in communication with at least one processor.

15. A non-invasive glucose monitoring apparatus according to claim 14 when appended to claim 12 or 13, wherein non-volatile storage and the at least one processor are housed within the housing.

16. A method for non-invasively monitoring blood glucose concentration in a subject, comprising:
transmitting, to an input of a microstrip conductor, an input signal, the microstrip conductor being arranged relative to a ground plane to define a space to receive a body part of the subject, the microstrip conductor and the ground plane together functioning as a microstrip transmission line having the body part of the subject as its substrate;
measuring an output signal from the microstrip transmission line;
determining at least one parameter of the output signal; and
determining, based on a comparison of the at least one parameter to at least one respective calibration curve, a glucose concentration of the user.

17. A method according to claim 16, wherein the at least one parameter comprises at least one parameter derived from the input impedance and/or the reflection coefficient.

18. A method according to claim 17, wherein the at least one parameter comprises one or more of: the real part of the input impedance, the imaginary part of the input impedance, the magnitude of the input impedance, the phase of the input impedance, the real part of the reflection coefficient, the imaginary part of the reflection coefficient, the magnitude of the reflection coefficient, and the phase of the reflection coefficient.

19. A method according to any one of claims 16 to 18, wherein the glucose concentration is determined based on a plurality of parameters derived from the output signal.
